(19) United States
(12) Patent Application Publication
(10) Pub. No.: US 2024/0065995 A1
(43) Pub. Date: Feb. 29, 2024

(54) INJECTABLE METHODS TO TREAT DEFICIENCIES IN GLUTATHIONE METABOLISM AND HOMOCYSTINURIA

(71) Applicant: Darren Alexander Rubin, Largo, FL (US)

(72) Inventor: Darren Alexander Rubin, Largo, FL (US)

(21) Appl. No.: 18/351,359

(22) Filed: Jul. 12, 2023

Related U.S. Application Data
(60) Provisional application No. 63/373,708, filed on Aug. 27, 2022.

(51) Int. Cl. A61K 31/198; A61K 9/00; A61K 47/18

(52) U.S. Cl. CPC A61K 31/198; A61K 9/0019; A61K 47/183

(57) ABSTRACT

Methods, formulations, and applications of increasing intracellular glutathione levels with at least one injectable cell membrane permeable prodrug or derivative of cysteine, glutamylcysteine, glutathione, or a combination thereof, to treat, reduce, or prevent cellular and tissue oxidative damage, to increase levels of amino acids, and to treat numerous disease states and conditions including inborn errors of metabolism, nutritional malabsorption, and aging.

### CROSS-REFERENCE TO RELATED APPLICATIONS

This application claims priority to U.S. Provisional Application Ser. No. 63/373,708 filed Aug. 27, 2022, entitled “Injectable Methods To Treat Deficiencies In Glutathione Metabolism And Homocystinuria,” the entire disclosure of which is hereby incorporated by reference in its entirety into the present disclosure.

### SPECIFICATION BACKGROUND

Cysteine is a conditionally essential amino acid, and the only proteinogenic amino acid having a sulfhydryl or thiol group. Cysteine is a source of sulfide for iron-sulfur clusters, including cofactors that participate in electron transfer for mitochondrial respiration. The thiol group of cysteinyl residues are often associated with other metal cofactors involved in important enzymatic activities. Cysteine residues often form disulfide bonds to provide vital roles in protein folding and protein stability. The amino acid taurine, which helps form bile salts and bile acids, is derived from cysteine.

Most healthy individuals produce cysteine from the essential amino acid methionine in what is known as the transsulfuration metabolic pathway. However, cysteine is considered a conditionally essential amino acid when enzymes in the transsulfuration pathway, such as cystathionase, are deficient or impaired, which can occur in patients with hepatic disease, in newborn infants requiring parenteral nutrition, and those with inborn errors of metabolism due to mutations in the gene(s) coding for such enzymes.

Disruption in the transsulfuration pathway leads to a condition known as homocystinuria. Classic homocystinuria is the inherited form of this disease, while vitamin B (e.g., B6, B12, B9) deficiencies can also lead to similar symptoms and disrupt methionine remethylation associated with this pathway. Homocysteine is a branch point between transsulfuration and methionine remethylation pathways. Inherited homocystinuria is caused by deficiency in the enzymes cystathionine-beta-synthase or cystathionase or methionine synthase. Homocystinuria can cause problems across the body, including central nervous system (CNS) abnormalities, skeletal abnormalities, eye abnormalities, as well as, vascular abnormalities, including blood clots.

Classic homocystinuria is often associated with markers of oxidative damage to lipids, proteins, and DNA throughout much of the body since the production of glutathione is impaired due to low levels of its precursor cysteine. Such patients have glutathione deficiency. Sometimes adding cysteine to the diet, even though cysteine may have low oral bioavailability, or administering L-cysteine injection, may help reduce this oxidative damage and lead to more glutathione production for patients with deficiencies in one or more enzymes of the transsulfuration pathway. But cysteine can oxidize to an insoluble dimer, cystine and can be toxic at high levels.

However, some individuals with glutathione deficiency have inborn errors of metabolism with mutations in genes encoding enzymes involved in the biosynthesis of glutathione, downstream of the transsulfuration pathway. In other words, these patients may be able to produce adequate cysteine, but may have an impaired ability to use that cysteine to create glutathione.

Glutathione is a tripeptide consisting of the amino acid residues cysteine, glutamate, and glycine. Cysteine combines with glutamic acid to form gamma-glutamylcysteine catalyzed by glutamate cysteine ligase (previously known as gamma-glutamylcysteine synthetase). Gamma-glutamylcysteine then combines with glycine to form glutathione catalyzed by glutathione synthase. Cysteine is believed the rate limiting factor in the formation of glutathione. If there is sufficient glutathione, glutathione may provide feedback inhibition on glutamate cysteine ligase.

Glutathione protects cells by neutralizing reactive oxygen species by participating in redox reactions with its thiol group of the cysteinyl residue. Glutathione has both a reduced and an oxidized state; a ratio of which may indicate a cell's level of oxidative stress. When neutralizing reactive oxygen species with the help of enzymes such as glutathione peroxidases, reduced glutathione oxidizes into a disulfide formed from two molecules of glutathione. The enzyme glutathione reductase with the coenzyme NADPH regenerates reduced glutathione from its oxidized disulfide state.

Oral bioavailability of glutathione is poor as it gets digested by proteases in the digestive tract. For glutathione deficient patients, one may think instead to inject glutathione directly into the patients' bloodstream. However, glutathione is not believed to be taken directly or readily into cells from freely circulating glutathione. There is believed no glutathione carrier or transporter for glutathione to be taken inside a cellular membrane; as glutathione is only believed to be exported out of cells. Yet, glutathione injection has become popular for cosmetic reasons with its antimelanogenic properties of skin lightening. Caution is advised, however, as glutathione intravenous injection may in some instances be associated with toxic effects on the liver, kidneys, and nervous system.

Glutathione in the bloodstream has a short half-life of under 2 minutes. Instead of entering cells, glutathione injected into the bloodstream gets degraded into its amino acids starting with the extracellular enzyme gamma-glutamyl transpeptidase, followed by gamma-glutamyl cyclotransferase. This forms 5-oxoproline and cysteinylglycine. The 5-oxoproline is converted to L-glutamate by 5-oxoprolinase, while cysteinylglycine is separated by dipeptidases into glycine and cysteine.

### DETAILED DESCRIPTION

The following description details methods of treating patients having glutathione deficiency and or thiol sensitive disorders with injectable precursors and prodrugs of glutathione and alternative sources of cysteine.

There exists a great need for increasing glutathione levels in individuals who are glutathione deficient or whose levels have declined with age and or disease. There also exists a great need for a safe and effective way to increase intracellular glutathione levels as glutathione injection does not directly or sufficiently increase intracellular levels of glutathione, and not without side effects. There also exists a great need to provide patients, including patients having homocystinuria or liver disease, with an alternative source of the amino acid cysteine. A solution is needed for providing methods of treating patients having glutathione deficiency and or thiol sensitive disorders with injectable precursors and prodrugs of glutathione and alternative sources of cysteine, including those that are cell membrane permeable. A solution is also needed to treat patients having malabsorption issues and other gastrointestinal problems that inhibit the patients' ability to adequately obtain amino acids, such as cysteine, and other glutathione precursors from the diet or from dietary supplements. Additionally, a solution is needed to efficiently increase intracellular levels of glutathione for a host of different conditions.

It was found that injection of certain derivatives of glutathione and or derivatives of its immediate precursor, gamma-glutamylcysteine, can enter cells from the bloodstream by crossing cellular membranes to increase intracellular glutathione levels in various organs and tissues of the body. In this sense, glutathione cell permeable glutathione derivatives act as prodrugs of glutathione which yield intracellular glutathione. The increase in intracellular glutathione can be relatively rapid, or can be controlled. Cell membranes are hydrophobic and generally allow small, electrically neutral molecules to pass. Glutathione esters; including glutathione monomethyl ester, glutathione monoethyl ester, glutathione diethyl ester, and glutathione isopropyl ester; represent one class of cell permeable glutathione derivatives. Synthetic esterification occurs on the carboxyl group of the glycine residue. The inventive methods allows different number of carbon atoms to permit different carbon chain lengths and branches esterified to the carboxyl group of the glycine residue. In some embodiments, the carbon chain may be less than 12 carbon atoms long. In some embodiments, the carbon chain may be up to 12 carbon atoms long. In some embodiments, the carbon chain may be 13 or more carbon atoms long. In some embodiments, the carbon chain may have two or multiple carbon chain branches and or two or multiple sets of carbon chain branches (i.e., be multi-branched). Other analogues and derivatives are possible and these examples are not meant to be limiting. Once inside the cell(s), these glutathione esters undergo hydrolysis into glutathione, thus increasing intracellular glutathione levels.

Other cell permeable glutathione derivatives include those with an acyl group, including S-acetyl-glutathione and other S-acyl-glutathione molecules. Longer and or larger acyl groups can also be utilized and these examples are not meant to be limiting. Another such cell permeable glutathione derivative is L-cysteine-glutathione as a disulfide. Other mixed glutathione disulfide molecules or derivatives are also possible. These examples are not meant to be limiting, and other derivatives and combinations exist to fulfill these needs. These prodrugs can treat patients with glutathione deficiency, especially those patients with inborn errors in metabolism that cannot sufficiently produce glutathione, i.e., those with ineffective or inefficient glutamate cysteine ligase and or glutathione synthase enzymes for which such treatments are able to bypass one or both of these enzymes.

The other molecules to enhance intracellular glutathione is membrane permeable derivatives of gamma-glutamylcysteine, including gamma-glutamylcysteinylethyl ester, which forms gamma-glutamylcysteine from intracellular esterases. Shorter, longer, and or branched ester carbon chains can be utilized and these examples are not meant to be limiting, and other derivatives and combinations exist to fulfill these needs. The dipeptide glutamylcysteine is the immediate precursor to glutathione that combines with glycine. For deficiencies in glutamate cysteine ligase, injection with gamma-glutamylcysteinylethyl ester bypasses that enzyme. For deficiencies in glutathione synthase, injection with gamma-glutamylcysteinylethyl ester can provide more substrate to help compensate and increase throughput for mutations coding for glutathione synthase having low enzyme efficiency. Additionally, the applicant's methods of injection of glutamylcysteine, cysteinylglycine, and or its derivatives are able to treat patients having malabsorption issues and other gastrointestinal problems that inhibit the patients' ability to adequately obtain cysteine, glutamate (and glutamine), and or glycine from the diet or from dietary supplements.

The present disclosure includes methods, formulations, and applications of providing non-oral sources of cysteine, glutamylcysteine, glutathione, or a combination thereof via prodrugs or derivatives of these small molecules, to patients, subjects, or individuals, preferably, but not limited to, injection. These patients, subjects, or individuals include those having one or more of the following conditions: low but not deficient cysteine levels, cysteine deficiency, low but not deficient glutamylcysteine levels, glutamylcysteine deficiency, low but not deficient glutathione levels, glutathione deficiency, homocystinuria, liver disease, other or related inborn errors of metabolism in one or more enzymes, amino acid or peptide oral malabsorption issues, an amino acid deficiency, and patients having ingested or received potentially hepatoxic quantity of acetaminophen. In further embodiments, the present disclosure provides cell permeable or cell membrane permeable prodrugs or derivatives of: cysteine for injection, glutamylcysteine for injection, and or glutathione for injection. Examples of such prodrug or derivative molecules include gamma-glutamylcysteinylethyl ester, S-acetyl-glutathione, glutathione diethyl ester, preferably for injection, but also for implantation.

In one embodiment, a formulation of the disclosure includes an aqueous solution of at least one cell membrane permeable prodrug or derivative of: cysteine, glutamylcysteine, glutathione, or a combination thereof; such as, but not limited to, an aqueous solution of gamma-glutamylcysteinylethyl ester, S-acetyl-glutathione, and or glutathione diethyl ester, preferably as an injectable solution. In this first embodiment, water is the only excipient ingredient; a solvent. However, the injectable solution preferably contains pH adjusters, such as sodium hydroxide and or hydrochloric acid. The concentration of the cell membrane permeable prodrug or derivative of cysteine, glutamylcysteine, and or, glutathione in the formulation can range from 0.1 mg/mL to over 50 mg/mL, and in some cases approach 100 mg/mL or more. By adjusting the pH and or using solubility enhancers, some embodiments have a concentration of the active ingredient approach, reach, or exceed 51 mg/mL. Alternatively, an injectable suspension embodiment or an implantable formulation embodiment may be desirable for maximizing potency and concentration, or providing a means of a supersaturated form of cell membrane permeable prodrug or derivative of cysteine, glutamylcysteine, glutathione, or a combination thereof.

In yet still other embodiments, the at least one injectable formulation of an at least one cell membrane permeable prodrug or derivative of cysteine, glutamylcysteine, glutathione, or a combination thereof is contained in a semi-solid or solid form, such as a capsule or tablet, or even a drug eluting stent or rod, such that gets implanted into a tissue or blood vessel. The solid form formulation(s) can be implanted manually by hand or by the use of an injector or autoinjector. The solid form will mix with and or dissolve in bodily fluid such as blood to impart its pharmacologic effect. In some embodiments, the one or more solid form formulation(s) is an implant. The implant can release drug immediately or slowly over time, and in this sense, the release and the increase in glutathione levels can be controlled.

Other pharmaceutically acceptable excipients or solvents may be included in the formulation, such as acids or bases to adjust pH, or salts to adjust osmolarity, preservatives, stabilizers, or even other solvents, carriers, and polymers. Example excipient ingredients may include glycerin, salts like sodium chloride and potassium bicarbonate, detergents and or chelators such as EDTA, certain polymers, pH adjustors (e.g., acids or bases), liposomes, preservatives, tonicity agents, solvents or water. Pharmaceutically acceptable excipients are preferred. These classes of excipients and or excipient examples are not meant to be limiting to one skilled in the art. It is desirable for the injectable formulation to be sterile. It is also desirable for the formulation to have good stability, low impurities, and a shelf life of at least six months, and preferably a shelf life of at least one year or more.

In many embodiments, the at least one formulation of an at least one cell membrane permeable prodrug or derivative of cysteine, glutamylcysteine, glutathione, or a combination thereof further comprises at least one excipient ingredient selected from solvents, tonicity agents, chelators, buffers, pH adjusting agents, solubility enhancing agents, preservatives, or a combination thereof. Water is an aqueous solvent. Various other solvents exist and can be employed as pharmaceutically acceptable solvents including organic and inorganic solvents, alcohols, e.g., ethanol, butanol, ketones, e.g., acetone, ethers, e.g., cyclopentyl methyl ether, oils, fatty acids and carboxylic acids, e.g., octanoic acid, solvent disulfides, and eutectic solvents. These examples are not meant to be limiting. Tonicity agents and salts can include sodium chloride and potassium chloride, and possibly sugars. These examples are not meant to be limiting. Chelators can include ethylenediaminetetraacetic acid (EDTA). These examples are not meant to be limiting. Buffers can include citric acid buffers, acetic acid buffers and sodium phosphate buffers. These examples are not meant to be limiting. Agents that adjust pH (pH adjusting agents) can include sodium hydroxide, acetic acid, hydrochloric acid, calcium carbonate, and gluconic acid. These examples are not meant to be limiting. Solubility enhancing agents can include polymers and liposomes. These examples are not meant to be limiting. Preservatives can include sodium benzoate, benzyl alcohol, metabisulfites, and chlorobutanol. These examples are not meant to be limiting. Various combinations of two or more of any of these excipients is possible and can be employed.

In some embodiments, the formulation includes only a single active ingredient of cell membrane permeable prodrug or derivative of cysteine, glutamylcysteine, or glutathione. In other embodiments, the formulation includes a combination of two, three, or more active ingredients of cell membrane permeable prodrug(s) or derivative(s) of cysteine, glutamylcysteine, or glutathione. In still further embodiments, the formulation includes at least one active ingredient of cell membrane permeable prodrug or derivative of cysteine, glutamylcysteine, glutathione, or a combination thereof; and can optionally include one or more additional active ingredients, including one or more additional active ingredients from different classes of drugs. In one such embodiment example, the formulation also includes betaine along with gamma-glutamylcysteinylethyl ester or S-acetyl-glutathione. Betaine is a methyl group donor that is involved in recycling homocysteine into the amino acid methionine in the methionine cycle with the enzyme betaine-homocysteine S-methyltransferase. This can be especially useful for patients with homocystinuria for decreasing levels of homocysteine. This also generates S-adenosyl methionine (SAM), which itself participates in many important methyl transfer reactions to nucleic acids, lipids, proteins, and other metabolites. In another embodiment example, the formulation also includes one or more vitamins. Preferably, one or more vitamins are vitamin B6, vitamin B12, vitamin B9, or a combination thereof. Such vitamins may serve as cofactors in these metabolic pathways to enhance treatment. Additional and or alternatively other vitamins, minerals, or other enzyme co-factors are also desirable. In another embodiment example, one or more of vitamin B3 and or other vitamins, minerals, nutrients, molecules, compounds, co-factors, coenzymes (e.g., coenzyme Q10), antioxidants, and or reducing agents, derivatives and or combinations thereof, that provide benefit, e.g., such as aiding in restoring redox balance, are included or alternatively included as an at least one additional ingredient. These examples are not meant to be limiting. In another embodiment example, the formulation also includes one or more an anti-inflammatory drugs, such as a nonsteroidal anti-inflammatory drug, e.g., ibuprofen, if the patient is experiencing inflammation. In another embodiment example, the formulation also includes at least one antibiotic or antiviral drug if the patient is experiencing an infection.

Methods of the present disclosure optionally include steps to measure levels of one or more of cysteine, glutamylcysteine, reduced glutathione, oxidized glutathione (and ratios of reduced glutathione:oxidized glutathione), and or metabolites thereof, in whole blood, blood plasma, blood cells, and or tissue samples; before treatment, during treatment, after treatment, or a combination thereof with one or more non-oral, preferably injectable, cell membrane permeable prodrug(s) or derivative(s) of cysteine, glutamylcysteine, glutathione, or a combination thereof. For example, whole blood can be tested for total glutathione levels with liquid chromatography/tandem mass spectrometry and or kinetic spectrophotometry, before, during, and or after treatment with injectable glutathione diethyl ester. Methods of the present disclosure optionally include steps to gene sequence or detect genetic mutations in, and or measure levels of, and or measure levels of activity of, one or more enzymes involved in and or related to the metabolism of cysteine, glutamylcysteine, glutathione, such as the transsulfuration pathway, the methionine remethylation pathway, glutathione synthesis pathway, or a combination above. Methods of the present disclosure also include steps to control and or titrate levels of intracellular glutathione and or its metabolites, up or down, based on repeated assay measurements.

Methods of the present disclosure include administering an at least one injection of an injectable formulation of an at least one cell membrane permeable prodrug or derivative of: cysteine, glutamylcysteine, and or glutathione. The administering of an at least one injection of an injectable formulation of an at least one cell membrane permeable prodrug or derivative of cysteine, glutamylcysteine, glutathione, or a combination thereof is selected from intravenous bolus injection, continuous intravenous infusion, subcutaneous injection, intramuscular injection, intrathecal injection, intraosseous injection, or implantation. The administering of an at least one injection of an injectable formulation of an at least one cell membrane permeable prodrug or derivative of cysteine, glutamylcysteine, glutathione, or a combination thereof is performed in some embodiments at least once per year, in other embodiments at least once per month, in other embodiments at least once per week, in other embodiments at least once per day, and in other embodiments at least once per hour.

Methods of the present disclosure include administering an at least second and or third dose, or more, of an at least one injection of an injectable formulation of an at least one cell membrane permeable prodrug or derivative of cysteine, glutamylcysteine, and or glutathione. The administering an at least second and or third dose, or more, of an at least one injection of an injectable formulation of an at least one cell membrane permeable prodrug or derivative of cysteine, glutamylcysteine, glutathione, or a combination thereof is selected from intravenous bolus injection, continuous intravenous infusion, subcutaneous injection, intramuscular injection, intrathecal injection, intraosseous injection, or implantation. The administering of an at least second and or third dose, or more, of an at least one injection of an injectable formulation of an at least one cell membrane permeable prodrug or derivative of cysteine, glutamylcysteine, glutathione, or a combination thereof is performed in some embodiments within a year, in other embodiments within a month, in other embodiments within a week, in other embodiments within a day, and in other embodiments within an hour, of the first dose. These methods of repeat dosing are not meant to be limited by these examples and other dosing regimens are possible within the scope of this disclosure.

In some embodiments, the injectable formulation of an at least one cell membrane permeable prodrug or derivative of cysteine, glutamylcysteine, glutathione, or a combination thereof is contained in a single-dose ampoule, vial, bottle, intravenous bag, prefilled syringe, cartridge, or autoinjector. In other embodiments, the injectable formulation of an at least one cell membrane permeable prodrug or derivative of cysteine, glutamylcysteine, glutathione, or a combination thereof is contained in a multi-dose ampoule, vial, bottle, intravenous bag, prefilled syringe, cartridge, or autoinjector. In many embodiments, the injectable formulation is a liquid. In some embodiments, the injectable formulation is a gel or semi-solid. In still other embodiments, the injectable formulation is in a concentrated and or dry form and or lyophilized powder form requiring dilution, reconstitution, or dissolution in a solvent (e.g., water for injection) prior to injection.

In some embodiments the autoinjector is powered by a spring mechanism. In other embodiments, the autoinjector is powered by compressed gas. In other embodiments, the liquid is contained in a cartridge, while in other embodiments the liquid is contained in a prefilled syringe. In other embodiments a prefilled syringe is administered manually. In yet still other embodiments, the prefilled syringe is administered with the aid of a syringe assist device that the prefilled syringe fits into, also known as a manual assist device. In some embodiments the injector contains a needle. In other embodiments the injector is needle-free and penetrates the skin with compressed gas or air.

In some embodiments, the formulation is delivered by a reusable pen or reusable autoinjector device associated with replaceable containers containing at least one cell membrane permeable prodrug or derivative of cysteine, glutamylcysteine, glutathione, or a combination thereof.

Some embodiments include administration with a portable infusion pump for prolonged subcutaneous, intramuscular, and or intravenous administration.

Other methods of administration include one or more intraosseous formulations and or one or more intraosseous delivery devices, while other methods of administration include one or more intrathecal formulations and or one or more intrathecal delivery devices.

In still further embodiments, the formulation of an at least one cell membrane permeable prodrug or derivative of cysteine, glutamylcysteine, glutathione, or a combination thereof is administered to blood, cells, and or blood cells taken outside the body, and then the treated blood, cells, and or blood cells are placed back inside the body. Such methods can ensure that blood cells have adequate amounts of glutathione, and may be helpful for treating certain diseases such as anemia, sickle cell anemia, and inborn errors of metabolism. When loading the blood, cells, and or blood cells with these molecules outside the body, changes in osmotic potential can be employed to help ensure uptake by the cells and or blood cells.

Other formats and or combinations of formulations and or other formats of delivery devices and or other routes of delivery of the at least one cell membrane permeable prodrug or derivative of cysteine, glutamylcysteine, glutathione, or a combination thereof are possible and the present disclosure is not intended to be limited to the embodiments shown herein, but is to be accorded the widest scope possible consistent with the principles and novel features as previously described.

Embodiments include immediate release formulations, while other embodiments include delayed or sustained release formulations.

At least one embodiment includes a formulation of glutamylcysteine as an active ingredient or nutritional supplement for injection. A liquid injectable formulation of glutamylcysteine can serve as an alternative source of cysteine or L-cysteine for injection, when broken down in the body. Glutamylcysteine can also serve as an alternative sources of glutamate when broken down in the body (and also as an alternative source of glutamine, since glutamate and glutamine can be interconverted in the body). Likewise, forms of glutathione can also serve as a source of the amino acids cysteine and glutamate (as well as glutamine). A liquid injectable formulation of gamma-glutamylcysteine can be indicated for use as an additive to amino acid solutions to meet the nutritional requirements of newborn infants requiring total parenteral nutrition, and of adult and pediatric patients with severe liver disease who may have impaired enzymatic processes and require total parenteral nutrition, and can also provide a more complete profile of amino acids for protein synthesis. The formulation may also treat acetaminophen overdose. The formulation may optionally include betaine and or one or more B vitamins selected from vitamin B6, vitamin B12, vitamin B3, and vitamin B9.

At least one embodiment includes a formulation of cysteinylglycine as an active ingredient or nutritional supplement for injection. A liquid injectable formulation of cysteinylglycine can serve as an alternative source of cysteine or L-cysteine for injection. A liquid injectable formulation of cysteinylglycine can be indicated for use as an additive to amino acid solutions to meet the nutritional requirements of newborn infants requiring total parenteral nutrition, and of adult and pediatric patients with severe liver disease who may have impaired enzymatic processes and require total parenteral nutrition, and can also provide a more complete profile of amino acids for protein synthesis. The formulation may also treat acetaminophen overdose. The formulation may optionally include betaine and or one or more B vitamins selected from vitamin B6, vitamin B12, vitamin B3, and vitamin B9.

While most of the embodiments are non-oral formulations, and preferably injectable formulations of an at least one cell membrane permeable prodrug or derivative of cysteine, glutamylcysteine, glutathione, or a combination thereof; or alternatively gamma-glutamylcysteine and or cysteinylglycine; oral formulations are possible, and includes, but is not limited to, oral capsules, oral tablets, oral liquids, oral suspensions, and oral pellets and oral sprinkles for mixing with liquid or food for drinking or eating. This also includes, but is not limited to, oral formulations with excipients where the active ingredients and or prodrugs are protected from peptidases and digestion in the gastrointestinal tract so that an adequate amount of active ingredient or prodrug is bioavailable intact in the blood stream and or liver. Such excipients may encapsulate or cage the molecule(s), thereby preventing access to the molecule(s) by enzymes and chemistry that would otherwise degrade them in the gastrointestinal tract. Enteral tube feeding formulations are also possible.

Some embodiments include delivery of an at least one cell membrane permeable prodrug or derivative of cysteine, glutamylcysteine, glutathione, or a combination thereof into cerebral spinal fluid and or inside the skull and or brain to protect and or treat the central nervous system.

In some embodiments, less that one milligram in an at least one injectable formulation of an at least one cell membrane permeable prodrug or derivative of cysteine, glutamylcysteine, glutathione, or a combination thereof is dosed. In other embodiments, more than one milligram in an at least one injectable formulation of an at least one cell membrane permeable prodrug or derivative of cysteine, glutamylcysteine, glutathione, or a combination thereof is dosed. In other embodiments, two or more milligrams in an at least one injectable formulation of an at least one cell membrane permeable prodrug or derivative of cysteine, glutamylcysteine, glutathione, or a combination thereof is dosed. In other embodiments, less than one gram in an at least one injectable formulation of an at least one cell membrane permeable prodrug or derivative of cysteine, glutamylcysteine, glutathione, or a combination thereof is dosed. In other embodiments, more than one gram in an at least one injectable formulation of an at least one cell membrane permeable prodrug or derivative of cysteine, glutamylcysteine, glutathione, or a combination thereof is dosed. In other embodiments, two or more grams in an at least one injectable formulation of an at least one cell membrane permeable prodrug or derivative of cysteine, glutamylcysteine, glutathione, or a combination thereof is dosed.

In some embodiments, less that one milliliter of an at least one injectable formulation of an at least one cell membrane permeable prodrug or derivative of cysteine, glutamylcysteine, glutathione, or a combination thereof is dosed. In other embodiments, more than one milliliter of an at least one injectable formulation of an at least one cell membrane permeable prodrug or derivative of cysteine, glutamylcysteine, glutathione, or a combination thereof is dosed. In other embodiments, two or more milliliters of an at least one injectable formulation of an at least one cell membrane permeable prodrug or derivative of cysteine, glutamylcysteine, glutathione, or a combination thereof is dosed. In other embodiments, less than one liter of an at least one injectable formulation of an at least one cell membrane permeable prodrug or derivative of cysteine, glutamylcysteine, glutathione, or a combination thereof is dosed. In other embodiments, more than one liter of an at least one injectable formulation of an at least one cell membrane permeable prodrug or derivative of cysteine, glutamylcysteine, glutathione, or a combination thereof is dosed. In other embodiments, two or more liters of an at least one injectable formulation of an at least one cell membrane permeable prodrug or derivative of cysteine, glutamylcysteine, glutathione, or a combination thereof is dosed.

In some embodiments, the method includes increasing intracellular levels of cysteine, glutamylcysteine, and or glutathione by more than 1 percent after dosage. In other embodiments, the method includes increasing intracellular levels of cysteine, glutamylcysteine, and or glutathione by two or more percent after dosage. In still other embodiments, the method includes increasing intracellular levels of cysteine, glutamylcysteine, and or glutathione by more than 10 percent after dosage. In still further embodiments, the method includes increasing intracellular levels of cysteine, glutamylcysteine, and or glutathione by more than 50 percent after dosage. In still further embodiments, the method includes increasing intracellular levels of cysteine, glutamylcysteine, and or glutathione by 100 percent after dosage. In still further embodiments, the method includes increasing intracellular levels of cysteine, glutamylcysteine, and or glutathione by more than 101 percent after dosage.

In some embodiments, the method includes maintaining an increased intracellular level of cysteine, glutamylcysteine, and or glutathione. Said method includes repeat administration of said injectable at least one cell membrane permeable prodrug or derivative of cysteine, glutamylcysteine, glutathione, or a combination thereof, such as to maintain increased levels over time; such as over an hour, or over a day, or over a week, or over a month or longer.

In some embodiments, the method includes increasing an intracellular level of cysteine, glutamylcysteine, and or glutathione after it has decreased, following a previous increase in intracellular level of cysteine, glutamylcysteine, and or glutathione. Said method includes repeat administration of said injectable at least one cell membrane permeable prodrug or derivative of cysteine, glutamylcysteine, glutathione, or a combination thereof.

In some embodiments, the method includes co-administering or subsequent administration of an at least one injectable formulation of an at least one cell membrane permeable prodrug or derivative of cysteine, glutamylcysteine, glutathione, or a combination thereof with an at least one gene therapy to correct an enzyme deficiency in one or more enzymes involved in and or related to the metabolism of cysteine, glutamylcysteine, glutathione, such as the transsulfuration pathway, the methionine remethylation pathway, glutathione synthesis pathway, or a combination above.

Embodiments of the inventive methods and formulations disclosed have the ability to treat, reduce, or prevent many medical conditions and disease states. Embodiments of the inventive methods and formulations disclosed also have the ability to increase and enhance general health and reduce the effects of aging and or stress. By protecting against oxidative damage and treating oxidative damage, embodiments can enhance the health and endurance of athletes and soldiers. Athletic applications exist whereby individuals or athletes receive an at least one injectable formulation of an at least one cell membrane permeable prodrug or derivative of cysteine, glutamylcysteine, glutathione, or a combination thereof before exercise or a competitive event, during exercise or a competitive event, and or after exercise or a competitive event, or a combination thereof, for better performance, reduced cellular or tissue damage, and enhanced recovery. Military applications also exist whereby soldiers, fighter pilots, or special forces or navy seals receive an at least one injectable formulation of an at least one cell membrane permeable prodrug or derivative of cysteine, glutamylcysteine, glutathione, or a combination thereof before a mission or combat, during a mission or combat, following a mission or combat, or a combination thereof. Such methods are believed to enhance performance and increase stamina and endurance. Other individuals may benefit from the inventive methods and formulations disclosed herein, and these examples are not meant to be limiting. The methods are also believed to enhance healing from a wound or injury and should be further evaluated.

Further embodiments include administering an at least one injectable formulation of an at least one cell membrane permeable prodrug or derivative of cysteine, glutamylcysteine, glutathione, or a combination thereof to treat, reduce, or prevent cellular oxidative damage and or tissue oxidative damage associated with oxygen gas toxicity in patients, scuba divers, pilots, and astronauts receiving supplemental oxygen gas administration and or nitrox gas administration, including from oxygen/gas tanks, rebreathers, pressurized chambers, pressurized suits, and or pressurized helmets. Therefore, there can also be navy, air force, space force, and NASA applications to these inventive methods.

Veterinary embodiments also exist and include administering an at least one injectable formulation of an at least one cell membrane permeable prodrug or derivative of cysteine, glutamylcysteine, glutathione, or a combination thereof to treat, reduce, or prevent disease or oxidative damage in a pet, zoo animal, livestock, or racehorse, and or to provide amino acid supplementation, and or increase intracellular glutathione levels. These animals are included as patients and or subjects in many embodiments.

Further embodiments include administering an at least one injectable formulation of an at least one cell membrane permeable prodrug or derivative of cysteine, glutamylcysteine, glutathione, or a combination thereof to treat, reduce, or prevent cellular oxidative damage and or tissue oxidative damage.

Further embodiments include administering an at least one injectable formulation of an at least one cell membrane permeable prodrug or derivative of cysteine, glutamylcysteine, glutathione, or a combination thereof to treat, reduce, or prevent in the body DNA oxidative damage, lipid oxidative damage, protein oxidative damage, or a combination thereof.

Further embodiments include administering an at least one injectable formulation of an at least one cell membrane permeable prodrug or derivative of cysteine, glutamylcysteine, glutathione, or a combination thereof to increase DNA repair.

Further embodiments include administering an at least one injectable formulation of an at least one cell membrane permeable prodrug or derivative of cysteine, glutamylcysteine, glutathione, or a combination thereof to repair and or restore mitochondrial function, mitochondrial respiration, and or increase the number of mitochondria. This can serve as treatment for one or more mitochondrial diseases and or improve a redox imbalance thereof.

Further embodiments include administering an at least one injectable formulation of an at least one cell membrane permeable prodrug or derivative of cysteine, glutamylcysteine, glutathione, or a combination thereof to reduce cellular apoptosis.

Further embodiments include administering an at least one injectable formulation of an at least one cell membrane permeable prodrug or derivative of cysteine, glutamylcysteine, glutathione, or a combination thereof to protect or prevent tissue damage from ischemia and or to treat tissue damage from ischemia; to protect or prevent tissue damage from surgery and or to treat tissue damage from surgery; to protect or prevent tissue damage from injury and or to treat tissue damage from injury; or a combination thereof.

Further embodiments include administering an at least one injectable formulation of an at least one cell membrane permeable prodrug or derivative of cysteine, glutamylcysteine, glutathione, or a combination thereof to treat, reduce, or prevent aging; treat, reduce, or prevent oxidative damage associated with aging; increasing levels of glutathione that decline with age; or a combination thereof.

Further embodiments include administering an at least one injectable formulation of an at least one cell membrane permeable prodrug or derivative of cysteine, glutamylcysteine, glutathione, or a combination thereof to treat infertility, maintain fertility, or prevent reduction in fertility.

Further embodiments include administering an at least one injectable formulation of an at least one cell membrane permeable prodrug or derivative of cysteine, glutamylcysteine, glutathione, or a combination thereof to treat, prevent, reduce or minimize toxicity and or nephrotoxicity associated with administration and or co-administration of chemotherapy drugs. An example of a chemotherapy drug is cisplatin.

Further embodiments include administering an at least one injectable formulation of an at least one cell membrane permeable prodrug or derivative of cysteine, glutamylcysteine, glutathione, or a combination thereof to treat, prevent, or reduce symptoms of scurvy.

Further embodiments include administering an at least one injectable formulation of an at least one cell membrane permeable prodrug or derivative of cysteine, glutamylcysteine, glutathione, or a combination thereof to treat, prevent, or reduce symptoms of anemia and or sickle cell anemia.

Further embodiments include administering an at least one injectable formulation of an at least one cell membrane permeable prodrug or derivative of cysteine, glutamylcysteine, glutathione, or a combination thereof to treat radiation exposure, or prevent or reduce or minimize damage from radiation, or prophylactically reduce or minimize damage from radiation.

Further embodiments include administering an at least one injectable formulation of an at least one cell membrane permeable prodrug or derivative of cysteine, glutamylcysteine, glutathione, or a combination thereof to treat, prevent, reduce, or minimize progression of at least one eye disease preferably selected from cataracts, glaucoma, macular degeneration, or a combination thereof.

Further embodiments include administering an at least one injectable formulation of an at least one cell membrane permeable prodrug or derivative of cysteine, glutamylcysteine, glutathione, or a combination thereof to treat, prevent, reduce, or minimize progression of hearing loss or inner ear disease.

Further embodiments include administering an at least one injectable formulation of an at least one cell membrane permeable prodrug or derivative of cysteine, glutamylcysteine, glutathione, or a combination thereof to protect neurons, nerves, and or the brain; and optionally treats/adjunctively treats neurological disease, including at least one of Alzheimer's disease, Parkinson's disease, amyotrophic lateral sclerosis, multiple sclerosis, traumatic brain injury, or a combination thereof. This may further optionally comprise delivery into cerebral spinal fluid, into the skull, into the brain, or a combination thereof.

Further embodiments include administering an at least one injectable formulation of an at least one cell membrane permeable prodrug or derivative of cysteine, glutamylcysteine, glutathione, or a combination thereof to influence, change, increase or curtail nitric oxide signaling; changing nitric oxide levels or bioavailability, or a combination thereof.

Further embodiments include administering an at least one injectable formulation of an at least one cell membrane permeable prodrug or derivative of cysteine, glutamylcysteine, glutathione, or a combination thereof to counter or neutralize toxic agents, venomous agents, poisonous agents, and biothreat agents. Some embodiments may thus serve as antidotes or adjunctive treatments for such exposures.

Further embodiments include administering an at least one injectable formulation of an at least one cell membrane permeable prodrug or derivative of cysteine, glutamylcysteine, glutathione, or a combination thereof to reduce viral or retroviral expression, infectivity, and or infection, and or reduce bacterial infectivity and or infection, or a combination thereof. In some instances, embodiments may also help reduce fungal and or yeast infectivity and or infection.

Further embodiments include administering an at least one injectable formulation of an at least one cell membrane permeable prodrug or derivative of cysteine, glutamylcysteine, glutathione, or a combination thereof to patients during and or following an infection to prevent, reduce, or treat cellular damage associated with inflammatory response, infection, or oxidative damage.

Further embodiments include administering an at least one injectable formulation of an at least one cell membrane permeable prodrug or derivative of cysteine, glutamylcysteine, glutathione, or a combination thereof to patients during and or following sepsis, septic shock, and or systemic inflammatory response syndrome to prevent, reduce, or treat cellular damage associated with inflammatory response, infection, or oxidative damage.

Further embodiments include administering an at least one injectable formulation of an at least one cell membrane permeable prodrug or derivative of cysteine, glutamylcysteine, glutathione, or a combination thereof to patients having or suffering from inflammatory conditions, including, but not limited to, lupus (e.g., systemic lupus) and or rheumatoid arthritis.

By definition, examples of an at least one “cell membrane permeable prodrug or derivative of cysteine, glutamylcysteine, and or glutathione” described in these embodiments above can include molecules and molecule classes such as glutathione esters; including glutathione monomethyl ester, glutathione monoethyl ester, glutathione diethyl ester, and glutathione isopropyl ester; glutathione derivatives with an acyl group, including S-acetyl-glutathione; and esters of gamma-glutamyl cysteine, including gamma-glutamylcysteinylethyl ester. These examples are not meant to be limiting.

Other formats and or combinations of formulations and or other formats of delivery devices and or other routes of delivery of an at least one formulation of an at least one cell membrane permeable prodrug or derivative of cysteine, glutamylcysteine, glutathione, or a combination thereof are possible and the present disclosure is not intended to be limited to the embodiments shown herein, but is to be accorded the widest scope possible consistent with the principles and novel features as previously described.

### Examples

Example 1 is an at least one injectable formulation of an at least one cell membrane permeable prodrug or derivative of: cysteine, glutamylcysteine, glutathione, or a combination thereof.

Example 2, the subject matter of Example 1 additionally or alternatively includes, wherein said at least one cell membrane permeable prodrug or derivative of cysteine, glutamylcysteine, glutathione, or a combination thereof comprises an at least one glutathione ester.

Example 3, the subject matter of any of Examples 1-2 additionally or alternatively includes, wherein said at least one cell membrane permeable prodrug or derivative of cysteine, glutamylcysteine, glutathione, or a combination thereof comprises an at least one S-acyl-glutathione.

Example 4, the subject matter of any of Examples 1-3 additionally or alternatively includes, wherein said at least one cell membrane permeable prodrug or derivative of cysteine, glutamylcysteine, glutathione, or a combination thereof comprises an at least one mixed glutathione disulfide molecule.

Example 5, the subject matter of any of Examples 1˜4 additionally or alternatively includes, wherein said at least one cell membrane permeable prodrug or derivative of cysteine, glutamylcysteine, glutathione, or a combination thereof comprises a gamma-glutamylcysteinyl ester.

Example 6, the subject matter of any of Examples 1-5 additionally or alternatively includes, wherein said at least one cell membrane permeable prodrug or derivative of cysteine, glutamylcysteine, glutathione, or a combination thereof comprises an at least one glutathione ester, S-acyl-glutathione, mixed glutathione disulfide molecule, gamma-glutamylcysteinyl ester, or a combination thereof.

Example 7, the subject matter of any of Examples 1-6 additionally or alternatively includes, wherein said at least one cell membrane permeable prodrug or derivative of cysteine, glutamylcysteine, glutathione, or a combination thereof comprises an at least one glutathione ester selected from glutathione monomethyl ester, glutathione monoethyl ester, glutathione diethyl ester, glutathione isopropyl ester, or a combination thereof; S-acyl-glutathione selected from S-acetyl-glutathione; mixed glutathione disulfide molecule selected from L-cysteine-glutathione as a disulfide; gamma-glutamylcysteinyl ester selected from gamma-glutamylcysteinylethyl ester; or a combination thereof.

Example 8, the subject matter of any of Examples 1-7 additionally or alternatively includes, further comprising an aqueous liquid solution.

Example 9, the subject matter of any of Examples 1-8 additionally or alternatively includes, further comprising at least one excipient ingredient in addition to water.

Example 10, the subject matter of any of Examples 1-9 additionally or alternatively includes, further comprising at least one excipient ingredient selected from solvents, tonicity agents, chelators, buffers, pH adjusting agents, solubility enhancing agents, preservatives, or a combination thereof.

Example 11, the subject matter of any of Examples 1-10 additionally or alternatively includes, further comprising at least one additional active pharmaceutical ingredient.

Example 12, the subject matter of any of Examples 1-11 additionally or alternatively includes, further comprising betaine.

Example 13, the subject matter of any of Examples 1-12 additionally or alternatively includes, further comprising at least one B vitamin selected from vitamin B6, vitamin B12, vitamin B9, vitamin B3, or a combination thereof. Additional and or alternatively other vitamins, minerals, or other enzyme co-factors are also desirable. Further, one or more of vitamin B3 and or other vitamins, minerals, molecules, compounds, co-factors, coenzymes (e.g., coenzyme Q10), antioxidants, and or reducing agents that provide benefit, e.g., such as aiding in restoring redox balance, are included or alternatively included as an at least one additional ingredient. This example is not meant to be limited.

Example 14, the subject matter of any of Examples 1-13 additionally or alternatively includes, further comprising at least one sterile container selected from prefilled syringes, cartridges, autoinjectors, vials, ampoules, intravenous fluid bags, and bottles.

Example 15, the subject matter of any of Examples 1-14 additionally or alternatively includes, further comprising a volume of at least 1 mL.

Example 16, the subject matter of any of Examples 1-15 additionally or alternatively includes, further comprising at least one milligram of said at least one cell membrane permeable prodrug or derivative of cysteine, glutamylcysteine, glutathione, or a combination thereof.

Example 17, the subject matter of any of Examples 1-16 additionally or alternatively includes, further comprising a concentration of at least one milligram per milliliter of said at least one cell membrane permeable prodrug or derivative of cysteine, glutamylcysteine, glutathione, or a combination thereof.

Example 18, the subject matter of any of Examples 1-17 additionally or alternatively includes, further comprising no more than 10% impurities at release or over its shelf life.

Example 19, the subject matter of any of Examples 1-18 additionally or alternatively includes, further comprising a shelf life of at least six months, and preferably a shelf life of at least one year or more.

Example 20, the subject matter of any of Examples 1-19 additionally or alternatively includes, further comprising a dry, lyophilized, or concentrated form requiring reconstitution or dilution with a solvent prior to use.

Example 21, the subject matter of any of Examples 1-20 additionally or alternatively includes, a method, comprising administering an at least one injectable formulation of an at least one cell membrane permeable prodrug or derivative of: cysteine, glutamylcysteine, glutathione, or a combination thereof to a patient or subject to increase intracellular glutathione levels.

Example 22, the subject matter of any of Examples 1-21 additionally or alternatively includes, wherein said method further increases intracellular glutathione levels by more than 1%, and preferably by more than 10%, and in some cases, by 50% or more. The increased level may be maintained over time; such as over an hour, or over a day, or over a week, or over a month or longer. This may include repeat injections in some instances.

Example 23, the subject matter of any of Examples 1-22 additionally or alternatively includes, wherein at least two or more injections of said an at least one injectable formulation of an at least one cell membrane permeable prodrug or derivative of cysteine, glutamylcysteine, glutathione, or a combination thereof are administered to a patient or subject. In some embodiments, there may be repeat injections; simultaneous injections, sequential injections, injections separated by time, and or injections separated by distance between injections sites.

Example 24, the subject matter of any of Examples 1-23 additionally or alternatively includes, wherein multiple injections of said an at least one injectable formulation of an at least one cell membrane permeable prodrug or derivative of cysteine, glutamylcysteine, glutathione, or a combination thereof are administered to a patient or subject. In some embodiments, there may be repeat injections; simultaneous injections, sequential injections, injections separated by time, and or injections separated by distance between injections sites.

Example 25, the subject matter of any of Examples 1-24 additionally or alternatively includes, wherein at least one injection of said an at least one injectable formulation of an at least one cell membrane permeable prodrug or derivative of cysteine, glutamylcysteine, glutathione, or a combination thereof is selected from intravenous bolus injection, continuous intravenous infusion, subcutaneous injection, intramuscular injection, intrathecal injection, intraosseous injection, autoinjection, or implantation.

Example 26, the subject matter of any of Examples 1-25 additionally or alternatively includes, further comprising the step of reconstituting or diluting said at least one injectable formulation of an at least one cell membrane permeable prodrug or derivative of cysteine, glutamylcysteine, glutathione, or a combination thereof with a solvent prior to use.

Example 27, the subject matter of any of Examples 1-26 additionally or alternatively includes, further comprising treating, reducing, or preventing cellular damage and tissue damage from oxidation.

Example 28, the subject matter of any of Examples 1-27 additionally or alternatively includes, further comprising treating, reducing, or preventing thiol sensitive disorders.

Example 29, the subject matter of any of Examples 1-28 additionally or alternatively includes, further comprising treating, preventing, or reducing symptoms associated with homocystinuria and or hepatic disease.

Example 30, the subject matter of any of Examples 1-29 additionally or alternatively includes, further comprising treating or reducing symptoms associated with inborn errors of metabolism and or enzyme deficiency and or enzyme inefficiency in at least one enzyme selected from glutamate cysteine ligase, glutathione synthase, cystathionine-beta-synthase, cystathionase, methionine synthase, or a combination thereof.

Example 31, the subject matter of any of Examples 1-30 additionally or alternatively includes, further comprising treating amino acid deficiencies in patients with peptide and or amino acid gastrointestinal malabsorption issues and related gastrointestinal conditions.

Example 32, the subject matter of any of Examples 1-31 additionally or alternatively includes, further comprising for use as an additive to amino acid solutions to meet the nutritional requirements of newborn infants requiring total parenteral nutrition, and of adult and pediatric patients with severe liver disease who may have impaired enzymatic processes and require total parenteral nutrition, and for providing a more complete profile of amino acids for protein synthesis.

Example 33, the subject matter of any of Examples 1-32 additionally or alternatively includes, further comprising delivery into cerebral spinal fluid and or inside the skull and or inside the brain to protect and or treat the central nervous system.

Example 34, the subject matter of any of Examples 1-33 additionally or alternatively includes, further comprising co-administering or subsequent administration of an at least one gene therapy to correct an enzyme deficiency in one or more enzymes involved in and or related to the metabolism of cysteine, glutamylcysteine, glutathione, such as the transsulfuration pathway, the methionine remethylation pathway, glutathione synthesis pathway, or a combination above.

Example 35, the subject matter of any of Examples 1-34 additionally or alternatively includes, further comprising reducing the effects of aging and or stress.

Example 36, the subject matter of any of Examples 1-35 additionally or alternatively includes, further comprising administration before exercise, combat, or a strenuous event; during exercise, combat, or a strenuous event; and or after exercise, combat, or a strenuous event; to provide at least one of enhanced performance, enhanced strength, enhanced stamina/endurance, improving recovery, reducing recovery time, enhancing healing, or a combination thereof.

Example 37, the subject matter of any of Examples 1-36 additionally or alternatively includes, wherein said patient or subject is a veterinary patient selected from a pet, a zoo animal, livestock, or a racehorse.

Example 38, the subject matter of any of Examples 1-37 additionally or alternatively includes, wherein said method further provides amino acid supplementation.

Example 39, the subject matter of any of Examples 1-38 additionally or alternatively includes, wherein said method further treats, prevents, or reduces in the body DNA oxidative damage, lipid oxidative damage, protein oxidative damage, or a combination thereof.

Example 40, the subject matter of any of Examples 1-39 additionally or alternatively includes, wherein said method further increases DNA repair.

Example 41, the subject matter of any of Examples 1-40 additionally or alternatively includes, wherein said method further repairs or restores mitochondrial function and or mitochondrial respiration; and or increases the number of mitochondria. This can serve as treatment for one or more mitochondrial diseases and or improve a redox imbalance thereof.

Example 42, the subject matter of any of Examples 1-41 additionally or alternatively includes, wherein said method further reduces cellular apoptosis.

Example 43, the subject matter of any of Examples 1-42 additionally or alternatively includes, wherein said method further protects or prevents tissue damage from ischemia and or treats tissue damage from ischemia; protects or prevents tissue damage from surgery and or treats tissue damage from surgery; protects or prevents tissue damage from injury and or treats tissue damage from injury; or a combination thereof.

Example 44, the subject matter of any of Examples 1-43 additionally or alternatively includes, wherein said method further treats, reduces, or prevents aging; treats, reduces, or prevents oxidative damage associated with aging; increases levels of glutathione that decline with age; or a combination thereof.

Example 45, the subject matter of any of Examples 1-44 additionally or alternatively includes, wherein said method further treats infertility, maintains fertility, or prevents reduction in fertility.

Example 46, the subject matter of any of Examples 1-45 additionally or alternatively includes, wherein said method further reduces or minimizes toxicity and or nephrotoxicity associated with administration and or co-administration of chemotherapy drugs.

Example 47, the subject matter of any of Examples 1-46 additionally or alternatively includes, wherein said method further treats, prevents, or reduces symptoms of scurvy.

Example 48, the subject matter of any of Examples 1-47 additionally or alternatively includes, wherein said method further treats, prevents, or reduces symptoms of anemia and or sickle cell anemia.

Example 49, the subject matter of any of Examples 1-48 additionally or alternatively includes, wherein said method further treats radiation exposure, or prevents or reduces or minimizes damage from radiation exposure, or prophylactically reduces or minimizes damage from radiation.

Example 50, the subject matter of any of Examples 1-49 additionally or alternatively includes, wherein said method further treats, prevents, or minimizes progression of at least one eye disease preferably selected from cataracts, glaucoma, macular degeneration, or a combination thereof.

Example 51, the subject matter of any of Examples 1-50 additionally or alternatively includes, wherein said method further treats, prevents, or minimizes progression of hearing loss or inner ear disease.

Example 52, the subject matter of any of Examples 1-51 additionally or alternatively includes, wherein said method further protects neurons, nerves, and or the brain. This method optionally treats/adjunctively treats neurological disease, including at least one of Alzheimer's disease, Parkinson's disease, amyotrophic lateral sclerosis, multiple sclerosis, traumatic brain injury, or a combination thereof. This may further optionally comprise delivery into cerebral spinal fluid, into the skull, into the brain, or a combination thereof.

Example 53, the subject matter of any of Examples 1-52 additionally or alternatively includes, wherein said method further changes nitric oxide levels or bioavailability, nitric oxide signaling, or a combination thereof.

Example 54, the subject matter of any of Examples 1-53 additionally or alternatively includes, wherein said method further treats/adjunctively treats neurological disease, including at least one of Alzheimer's disease, Parkinson's disease, amyotrophic lateral sclerosis (ALS), traumatic brain injury, or a combination thereof.

Example 55, the subject matter of any of Examples 1-54 additionally or alternatively includes, wherein said method further treats/adjunctively treats or counters exposure to toxic agents, venomous agents, poisonous agents, and biothreats; and may thus serve as antidotes or adjunctive treatments.

Example 56, the subject matter of any of Examples 1-55 additionally or alternatively includes, wherein said method further reduces viral or retroviral expression, infectivity, and or infection, and or reduces bacterial infectivity and or infection, or a combination thereof.

Example 57, the subject matter of any of Examples 1-56 additionally or alternatively includes, wherein said method further prevents or treats cellular damage associated with inflammatory response, infection, or oxidative damage, including that associated with sepsis, septic shock, and or systemic inflammatory response syndrome.

Example 58, the subject matter of any of Examples 1-57 additionally or alternatively includes, wherein said method further treats/adjunctively treats inflammatory conditions, including lupus (e.g., systemic lupus) and or rheumatoid arthritis.

Example 59, the subject matter of any of Examples 1-58 additionally or alternatively includes, wherein said method further treats and or prophylactically prevents oxidative damage associated with oxygen gas toxicity in patients, scuba divers, pilots, and astronauts receiving supplemental oxygen gas administration and or nitrox gas administration, including from oxygen/gas tanks (including their associated masks, nasal cannulas, and mouthpieces), rebreathers, pressurized chambers, pressurized suits, and or pressurized helmets. Therefore, there can also be navy, air force, space force, and NASA applications to these inventive methods.

Example 60, the subject matter of any of Examples 1-59 additionally or alternatively includes, wherein said method further includes at least one step of measuring levels of one or more of cysteine, glutamylcysteine, reduced glutathione, oxidized glutathione, and or metabolites thereof, in whole blood, blood plasma, blood cells, and or tissue samples; before said administration, during said administration, after said administration, or a combination thereof, of said at least one injectable formulation of an at least one cell membrane permeable prodrug or derivative of cysteine, glutamylcysteine, glutathione, or a combination thereof.

Example 61, the subject matter of any of Examples 1-60 additionally or alternatively includes, wherein said method further includes at least one step of gene/genetic sequencing or detecting genetic mutations in, and or measuring levels of, and or measuring levels of activity of, one or more enzymes involved in and or related to the metabolism of cysteine, glutamylcysteine, glutathione, such as the transsulfuration pathway, the methionine remethylation pathway, glutathione synthesis pathway, or a combination thereof; before said administration, during said administration, after said administration, or a combination thereof, of said at least one injectable formulation of an at least one cell membrane permeable prodrug or derivative of cysteine, glutamylcysteine, glutathione, or a combination thereof.

Example 62, the subject matter of any of Examples 1-61 additionally or alternatively includes, wherein said method further includes steps to control and or titrate levels of intracellular glutathione and or its metabolites, up or down, based on repeated assay measurements.

Example 63, the subject matter of any of Examples 1-62 additionally or alternatively includes, wherein said method further enhances healing from a wound or injury.

Still further embodiments and examples are disclosed herein.

Example 64, is an at least one injectable formulation of glutamylcysteine, cysteinylglycine, or a combination thereof.

Example 65, the subject matter of Example 64 additionally or alternatively includes, a method of administering an at least one injectable formulation of glutamylcysteine, cysteinylglycine, or a combination thereof to provide an increase in levels of cysteine and or glutathione. This method may also treat an otherwise hepatoxic acetaminophen overdose. Glutamylcysteine can also provide an increase in levels of glutamate and glutamine when metabolized.

Example 66, the subject matter of any of Examples 64-65 additionally or alternatively includes, a method of administering an at least one injectable formulation of glutamylcysteine, cysteinylglycine, or a combination thereof for use as an additive to amino acid solutions to meet the nutritional requirements of newborn infants requiring total parenteral nutrition, and of adult and pediatric patients with severe liver disease who may have impaired enzymatic processes and require total parenteral nutrition, and for providing a more complete profile of amino acids for protein synthesis.

The injectable formulations described above, along with methods of administering these injectable formulations, are different from, and not intended to include, regular forms of 1-cysteine, 1-glutamine, or N-acetyl cysteine (NAC), which are readily commercially available.

In the embodiments and examples described above, formulations, including injectable formulations, are generally meant to be pharmaceutically acceptable, and as such, are generally safe, non-toxic, and therapeutically desirable for use in human and or veterinary patients.

Various illustrative components, blocks, configurations, modules, and steps have been described above generally in terms of their functionality. Persons having ordinary skill in the art may implement the described functionality in varying ways for each particular application, but such implementation decisions should not be interpreted as causing a departure from the scope of the present disclosure.

The previous description of the disclosed embodiments is provided to enable a person skilled in the art to make or use the disclosed embodiments. Various modifications to these embodiments will be readily apparent to those skilled in the art, and the principles defined herein may be applied to other embodiments without departing from the scope of the disclosure. Thus, the present disclosure is not intended to be limited to the embodiments shown herein but is to be accorded the widest scope possible consistent with the principles and novel features as previously described.

What is claimed is:

1. An at least one injectable formulation of an at least one cell membrane permeable prodrug or derivative of cysteine, glutamylcysteine, glutathione, or a combination thereof.

2. The at least one injectable formulation of an at least one cell membrane permeable prodrug or derivative of cysteine, glutamylcysteine, glutathione, or a combination thereof of claim 1 wherein said at least one cell membrane permeable prodrug or derivative of cysteine, glutamylcysteine, glutathione, or a combination thereof comprises an at least one glutathione ester, S-acyl-glutathione, mixed glutathione disulfide molecule, gamma-glutamylcysteinyl ester, or a combination thereof.

3. The at least one injectable formulation of an at least one cell membrane permeable prodrug or derivative of cysteine, glutamylcysteine, glutathione, or a combination thereof of claim 1 further comprising an aqueous liquid solution.

4. The at least one injectable formulation of an at least one cell membrane permeable prodrug or derivative of cysteine, glutamylcysteine, glutathione, or a combination thereof of claim 1 further comprising at least one excipient ingredient selected from solvents, tonicity agents, chelators, buffers, pH adjusting agents, solubility enhancing agents, preservatives, or a combination thereof.

5. The at least one injectable formulation of an at least one cell membrane permeable prodrug or derivative of cysteine, glutamylcysteine, glutathione, or a combination thereof of claim 1 further comprising at least one sterile container selected from prefilled syringes, cartridges, autoinjectors, vials, ampoules, intravenous fluid bags, and bottles.

6. The at least one injectable formulation of an at least one cell membrane permeable prodrug or derivative of cysteine, glutamylcysteine, glutathione, or a combination thereof of claim 1 further comprising an amount of at least one milligram, a volume of at least one milliliter, a concentration of at least one milligram per milliliter, or a combination thereof, of said at least one cell membrane permeable prodrug or derivative of cysteine, glutamylcysteine, glutathione, or a combination thereof.

7. The at least one injectable formulation of an at least one cell membrane permeable prodrug or derivative of cysteine, glutamylcysteine, glutathione, or a combination thereof of claim 1 further comprising no more than 10% impurities at release and or over its shelf life.

8. The at least one injectable formulation of an at least one cell membrane permeable prodrug or derivative of cysteine, glutamylcysteine, glutathione, or a combination thereof of claim 1 further comprising a dry, lyophilized, or concentrated form requiring reconstitution or dilution with a solvent prior to use.

9. A method, comprising:
administering an at least one injectable formulation of an at least one cell membrane permeable prodrug or derivative of cysteine, glutamylcysteine, glutathione, or a combination thereof to a patient or subject to increase intracellular glutathione levels.

10. The method of claim 9, wherein the intracellular glutathione levels are increased by more than 1%.

11. The method of claim 9, wherein the at least one injectable formulation of an at least one cell membrane permeable prodrug or derivative of cysteine, glutamylcysteine, glutathione, or a combination thereof comprises an at least one glutathione ester, S-acyl-glutathione, mixed glutathione disulfide molecule, gamma-glutamylcysteinyl ester, or a combination thereof.

12. The method of claim 9, further comprising administering at least two or more injections of said an at least one injectable formulation of an at least one cell membrane permeable prodrug or derivative of cysteine, glutamylcysteine, glutathione, or a combination thereof to a patient or subject.

13. The method of claim 12, further comprising selecting at least one injection of said at least two or more injections is from intravenous bolus injection, continuous intravenous infusion, subcutaneous injection, intramuscular injection, intrathecal injection, intraosseous injection, autoinjection, or implantation.

14. The method of claim 9, further comprising reconstituting or diluting said at least one injectable formulation of an at least one cell membrane permeable prodrug or derivative of cysteine, glutamylcysteine, glutathione, or a combination thereof with a solvent prior to use.

15. The method of claim 9, further comprising administering the at least one injectable formulation to:
treat, prevent, or reduce cellular damage and tissue damage from oxidation; treating, preventing, or reducing cellular damage and tissue damage from ischemia;
treat, prevent, or reduce cellular damage and tissue damage from surgery; treating, preventing, or reducing cellular damage and tissue damage from injury; or
treat, prevent, or reduce the effects of stress.

16. The method of claim 9, further comprising administering the at least one injectable formulation to treat, prevent, or reduce aging and or symptoms of aging; treats, reduces, or prevents oxidative damage associated with aging; increases levels of glutathione that decline with age; or a combination thereof.

17. The method of claim 9, further comprising administering the at least one injectable formulation to:
treat, prevent, or reduce thiol sensitive disorders; or
treat, prevent, or reduce symptoms associated with homocystinuria and or hepatic disease.

18. The method of claim 9, further comprising administering the at least one injectable formulation to treat or reduce symptoms associated with inborn errors of metabolism and or enzyme deficiency and or enzyme inefficiency in at least one enzyme selected from glutamate cysteine ligase, glutathione synthase, cystathionine-beta-synthase, cystathionase, methionine synthase, or a combination thereof.

19. The method of claim 18, further comprising gene/genetic sequencing or detecting genetic mutations in, and or measuring levels of, and or measuring levels of activity of, one or more enzymes involved in and or related to the metabolism of cysteine, glutamylcysteine, glutathione, or a combination thereof.

20. The method of claim 9, further comprising measuring levels of one or more of cysteine, glutamylcysteine, reduced glutathione, oxidized glutathione, and or metabolites thereof, in whole blood, blood plasma, blood cells, and or tissue samples; before said administration, during said administration, after said administration, or a combination thereof, of said at least one injectable formulation of an at least one cell membrane permeable prodrug or derivative of cysteine, glutamylcysteine, glutathione, or a combination thereof.

21. The method of claim 9, further comprising administering the at least one injectable formulation to treat amino acid deficiencies in patients with peptide and or amino acid gastrointestinal malabsorption issues and related gastrointestinal conditions, and or providing amino acid supplementation.

22. The method of claim 9, further comprising administering the at least one injectable formulation for use as an additive to amino acid solutions to meet the nutritional requirements of newborn infants requiring total parenteral nutrition, and or of adult and pediatric patients with severe liver disease who may have impaired enzymatic processes and require total parenteral nutrition, and or for providing a more complete profile of amino acids for protein synthesis, and or for treating an otherwise hepatoxic acetaminophen overdose.

23. The method of claim 9, further comprising administering the at least one injectable formulation before exercise, combat, or a strenuous event; during exercise, combat, or a strenuous event; and or after exercise, combat, or a strenuous event; to provide at least one of enhanced performance, enhanced strength, enhanced stamina/endurance, improving recovery, reducing recovery time, enhancing healing, or a combination thereof.

24. The method of claim 9, further comprising administering the at least one injectable formulation to treat, prevent, or reduce in the body DNA oxidative damage, lipid oxidative damage, protein oxidative damage, or a combination thereof.

25. The method of claim 9, further comprising administering the at least one injectable formulation to increase DNA repair, enhance healing from a wound or injury, reduce cellular apoptosis, reduce impaired fertility, or a combination thereof.

26. The method of claim 9, further comprising administering the at least one injectable formulation to repair or restore mitochondrial function and or mitochondrial respiration; and/or increase the number of mitochondria; and/or treat at least one mitochondrial disease and or improve a redox imbalance thereof.

27. The method of claim 9, further comprising administering the at least one injectable formulation to treat, prevent, or reduce damage from radiation exposure; treat, prevent, or reduce toxicity associated with chemotherapy; or a combination thereof.

28. The method of claim 9, further comprising administering the at least one injectable formulation to at least one of treat, prevent, or reduce symptoms of scurvy; treat, prevent, or reduce symptoms of anemia and or sickle cell anemia; treat, prevent, or reduce eye disease; or treat, prevent, or reduce hearing loss or inner ear disease.

29. The method of claim 9, wherein administering the at least one injectable formulation causes changes in nitric oxide levels or bioavailability, nitric oxide signaling, or a combination thereof.

30. The method of claim 9, wherein administering the at least one injectable formulation protects neurons, nerves, and/or the brain.

31. The method of claim 30, further comprising administering the at least one injectable formulation to treat/adjunctively treat neurological disease, including at least one of Alzheimer's disease, Parkinson's disease, amyotrophic lateral sclerosis, multiple sclerosis, traumatic brain injury, or a combination thereof.

32. The method of claim 30, further comprising administering the at least one injectable formulation via delivery into cerebral spinal fluid, into the skull, into the brain, or a combination thereof.

33. The method of claim 9, wherein administering the at least one injectable formulation reduces viral or retroviral expression, infectivity, and or infection, and or reduces bacterial infectivity and or infection, or a combination thereof.

34. The method of claim 9, wherein administering the at least one injectable formulation treats at least one inflammatory condition selected from sepsis, septic shock, systemic inflammatory response syndrome, lupus, or rheumatoid arthritis.

35. The method of claim 9, wherein administering the at least one injectable formulation treats and or prophylactically prevents oxidative damage associated with oxygen gas toxicity in patients, scuba divers, pilots, and astronauts receiving supplemental oxygen gas administration and or nitrox gas administration.

36. The method of claim 9, wherein administering the at least one injectable formulation treats/adjunctively treats or counters exposure to toxic agents, venomous agents, poisonous agents, and biothreat agents.

37. An at least one injectable formulation of glutamylcysteine, cysteinylglycine, or a combination thereof.

38. A method of administering an at least one injectable formulation of glutamylcysteine, cysteinylglycine, or a combination thereof for use as an additive to amino acid solutions to meet the nutritional requirements of newborn infants requiring total parenteral nutrition, and or of adult and pediatric patients with severe liver disease who may have impaired enzymatic processes and require total parenteral nutrition, and or for providing a more complete profile of amino acids for protein synthesis, and or for treating an otherwise hepatoxic acetaminophen overdose.
